(19) United States
(12) Patent Application Publication
(10) Pub. No.: US 2015/0024434 A1
(43) Pub. Date: Jan. 22, 2015

(54) METHOD FOR OBTAINING FAB FRAGMENTS FROM SINGLE ANTIBODY PRODUCING CELLS BY MULTIPLEXED CPR IN COMBINATION WITH TaqMan PROBES

(71) Applicant: HOFFMANN-LA ROCHE INC., Nutley, NJ (US)

(72) Inventors: Hans-Willi Krell, Penzberg (DE); Alexander Lifke, Penzberg (DE); Valeria Lifke, Penzberg (DE); Kairat Madin, Penzberg (DE); Christian Weilke, Penzberg (DE)

(21) Appl. No.: 14/346,705

(22) PCT Filed: Sep. 20, 2012
(86) PCT No.: PCT/EP2012/068532

§ 371 Date: Mar. 21, 2014

(30) Foreign Application Priority Data
Sep. 21, 2011 (EP) 11182223.5

(51) Int. Cl. C12Q 1/68; C07K 16/00

(52) U.S. Cl. CPC C12Q 1/6851; C07K 16/00; C07K 2317/10
USPC 435/69.6; 506/9; 536/24.33; 435/91.21

(57) ABSTRACT

Herein is reported a method for a multiplex one tube real-time reverse-transcriptase gene-specific polymerase chain reaction for the amplification and quantification of cognate IgG heavy and light chains encoding nucleic acids (human IgG isotype) from a single cell.

Herein is reported a method for obtaining antibodies from single antibody producing cells by the combination of a multiplexed polymerase chain reaction (PCR) and TaqMan probes in order to allow for rapid screening of PCR products. The Fab fragments of the respective antibodies can be obtained by in vitro translation and the binding properties of the Fab fragments can determined.

### BACKGROUND OF THE INVENTION

Since the establishment of hybridoma technology (Cole, S. P. C., et al., Monoclonal Antibodies and Cancer Therapy, Alan R. Liss, p. 77 (1985); and Boerner, P., et al., J. Immunol. 147 (1991) 86-95), monoclonal immunoglobulins have emerged to play a pivotal role in scientific research, human healthcare and diagnostics. Consequently, the generation of monoclonal, especially therapeutic, immunoglobulins is a field undergoing intensive research. In this respect, the hybridoma technology and phage display technology (Hoogenboom, H. R., and Winter, G., J. Mol. Biol. 227 (1992) 381-388; Marks, J. D., et al., J. Mol. Biol. 222 (1991) 581-597) are, amongst others, two commonly used technologies for the generation of monoclonal immunoglobulins. In hybridoma technology obtaining of stable clones is a hurdle, thus, diminishing diversity of the antibodies, as only a limited number of B-cells are successfully fused, propagated and thereafter characterized. Similarly, a drawback of phage or yeast display-based combinatorial library approaches is the random pairing of the immunoglobulin heavy and light chains. The dissociation of the original heavy and light chain pairing, and non-cognate pairing, necessitate the screening of a large number of immunoglobulin producing cells in order to identify heavy and light chain pairs of high affinity. In addition, such non-cognate pairs may display unwanted cross-reactivity to human antigens. Finally, the genetic diversity of target-specific immunoglobulins identified by selection and screening of combinatorial libraries is commonly limited due to inherent selection biases.

Generation of immunoglobulins from immunoglobulin producing cell can be performed according to methods known in the art. Such methods are e.g. hybridoma technique. A different method is based on the identification of the nucleic acid sequence of the immunoglobulin. Usually it is sufficient to identify the sequence of the variable regions or even only the CDR regions or only the CDR3 region. For example, the mRNA is isolated from a pool of immunoglobulin producing cells and is used for the construction of a cDNA-library encoding the CDR regions of the immunoglobulin. The cDNA-library is then transfected into a suitable host cell, such as NS0 or CHO, and screened for specific immunoglobulin production.

WO 2008/104184 reports a method for cloning cognate antibodies. The efficient generation of monoclonal antibodies from single human B cells is reported by Tiller et al. (Tiller, T., et al., J. Immunol. Meth. 329 (2007) 112-124). Braeuninger et al. (Braeuninger, A., et al., Blood 93 (1999) 2679-2687) report the molecular analysis of single B cells from T-cell-rich B-cell lymphoma. Systematic design and testing of nested (RT-) PCR primer is reported by Rohatgi et al. (Rohatgi, S., et al, J. Immunol. Meth. 339 (2008) 205-219). In WO 02/13862 a method and composition for altering a B-cell mediated pathology are reported. Haurum et al. (Meijer, P. J. and Haurum, J. S., J. Mol. Biol. 358 (2006) 764-772) report a one-step RT-multiplex overlap extension PCR. Stollar et al. and Junghans et al. report the sequence analysis by single cell PCR reaction (Wang, X. and Stollar, B. D., J. Immunol. Meth. 244 (2000) 217-225; Coronella, J. A. and Junghans, R. P., Nucl. Acids Res. 28 (2000) E85). Jiang, X. and Nakano, H., et al. (Biotechnol. Prog. 22 (2006) 979-988) report the construction of a linear expression element for in vitro transcription and translation.

### SUMMARY OF THE INVENTION

It has been found that the generally used multi-step approaches for obtaining cognate VH and VL encoding nucleic acids can be improved (to be e.g. more rapid and robust) by combining the required primers for a reverse transcription and gene specific polymerase chain reaction and the probes required for real-time quantification in a multiplex one tube real-time polymerase chain reaction.

Herein is reported as an aspect a method for a multiplex one tube real-time reverse-transcriptase gene-specific polymerase chain reaction for the amplification and quantification of cognate IgG heavy and light chains encoding nucleic acids (human IgG isotype) from a single B-cell or plasmablast or plasma cell comprising the following step:
performing a reverse transcription and polymerase chain reaction in one step with a first and a second 5′-primer and a first and a second 3′-primer and a first and a second TaqMan probe.

In one embodiment the first 5′-primer is complementary to a nucleic acid sequence encoding the heavy chain leader peptide or the first heavy chain framework region. In one embodiment the second 5′-primer is complementary to a nucleic acid sequence encoding the light chain leader peptide or the first light chain framework region. In one embodiment the first 3′-primer is complementary to a nucleic acid sequence encoding the C-terminal amino acid residues of a heavy chain CH1 domain. In one embodiment the second 3′-primer is complementary to a nucleic acid sequence encoding the C-terminal amino acid residues of a light chain constant domain. In one embodiment the first TaqMan probe is complementary to a nucleic acid encoding N-terminal amino acid residues of a heavy chain CH1 domain. In one embodiment the second TaqMan probe is complementary to a nucleic acid encoding N-terminal amino acid residues of a light chain constant domain.

Herein is also reported as one aspect a method for obtaining a monoclonal antibody comprising the in vitro translation of a nucleic acid encoding human immunoglobulin G fragments whereby the nucleic acid is obtained by specific amplification of cDNA fragments obtained from the mRNA of a single immunoglobulin producing human B-cell, plasmablast or plasma cell or a B-cell of an animal comprising a human immunoglobulin locus with a method for a multiplex one tube real-time reverse-transcriptase gene-specific polymerase chain reaction for the amplification and quantification of cognate IgG heavy and light chain encoding nucleic acids as reported herein.

In one embodiment the Fab PCR product is subsequently transcribed to mRNA and translated in vitro employing E. coli lysate.

With the methods as reported herein it is possible to characterize a multitude of provided B-cells with respect to the antigen binding characteristics of their produced immunoglobulin. Thus, no loss of immunoglobulin diversity occurs. As the analyzed B-cells are mature B-cells obtained after the in vivo maturation process it is very unlikely that their produced immunoglobulins show cross-reactivity with other antigens.

In a further embodiment the methods as reported herein are characterized in that the primer provide for overhangs encoding the translational start codon ATG for 5′-primer and/or the translational stop codon TTA for 3′-primer. In still a further embodiment the methods as reported herein are characterized in comprising the additional step of:
providing a single cell and obtaining the mRNA of this cell.

A further aspect as reported herein is a method for producing an immunoglobulin Fab-fragment comprising the following steps:
providing a single immunoglobulin producing cell,
obtaining from the cell the nucleic acid encoding the immunoglobulin light and heavy chain variable domains, optionally also encoding a part of the light chain constant domain and a part of the heavy chain C_{H}1 domain with a multiplex one tube real-time reverse-transcriptase gene-specific polymerase chain reaction for the amplification and quantification of cognate IgG heavy and light chains encoding nucleic acids as reported herein,
generating a linear expression matrix comprising the obtained nucleic acid,
translating in vitro the nucleic acid and thereby producing the immunoglobulin Fab fragment.

Another aspect as reported herein is a method for producing an immunoglobulin comprising the following steps:
providing a single immunoglobulin producing cell,
obtaining from the cell the nucleic acid encoding the immunoglobulin light and heavy chain variable domains with a multiplex one tube real-time reverse-transcriptase gene-specific polymerase chain reaction for the amplification and quantification of cognate IgG heavy and light chains encoding nucleic acids as reported herein,
operably linking each of the nucleic acids obtained in the previous step with a nucleic acid encoding the not encoded C-terminal constant domain amino acid residues of the respective immunoglobulin light or heavy chain constant domain,
transfecting a eukaryotic or a prokaryotic cell with the nucleic acids obtained in the previous step,
cultivating the transfected cell, in one embodiment under conditions suitable for the expression of the immunoglobulin,
recovering the immunoglobulin from the cell or the cultivation medium and thereby producing an immunoglobulin.

In one embodiment of all methods as reported herein is the immunoglobulin an immunoglobulin of class G (IgG).

In one embodiment of all methods as reported herein each of the primer is independently of each other selected from the group comprising SEQ ID NO: 05, SEQ ID NO: 06, SEQ ID NO: 07, SEQ ID NO: 08, SEQ ID NO: 09, SEQ ID NO: 10, SEQ ID NO: 11, SEQ ID NO: 12.

In one embodiment of all methods as reported herein the polymerase chain reaction is performed with a pair of primer independently of each other selected from the group comprising SEQ ID NO: 05, SEQ ID NO: 06, SEQ ID NO: 07, SEQ ID NO: 08, SEQ ID NO: 09, SEQ ID NO: 10, SEQ ID NO: 11, SEQ ID NO: 12.

### DESCRIPTION OF THE INVENTION

Herein is reported as an aspect a method for a multiplex one tube real-time reverse-transcriptase gene-specific polymerase chain reaction for the amplification and quantification of cognate IgG heavy and light chains encoding nucleic acids (human IgG isotype) from a single B-cell or plasmablast or plasma cell comprising the following step:
performing a reverse transcription and polymerase chain reaction in one step with a first and a second 5′-primer and a first and a second 3′-primer and a first and a second TaqMan probe.

It has been found that the generally used multi-step approaches for obtaining cognate VH and VL encoding nucleic acids can be improved (to be e.g. more rapid and robust) by combining the required primers for a reverse transcription and gene specific polymerase chain reaction and the probes required for real-time quantification in a multiplex one tube real-time polymerase chain reaction.

Such an approach is especially useful as other possible ways to improve the currently used two step methods have certain drawbacks. For example, a high primer concentration to increase sensitivity is not suited due to the possible induction of primer-primer-dimer formation and/or the induction of non-specific binding, or increasing the number of amplification cycles can result in the amplification of non-specific sequences.

By employing magnetic micro-beads coated with the human pan B-cell marker, CD19 (see e.g. Bertrand, F. E., III, et al., Blood 90 (1997) 736-744), B-cells can be isolated from peripheral blood. With the limited dilution approach, single cells can be placed in the wells of 96 well microtiter plate. The mRNA of these cells can be extracted.

In the methods as reported herein a multiplex polymerase chain reaction is used for the amplification of heavy and light chain variable domain encoding nucleic acids simultaneously in a one tube polymerase chain reaction. In contrast to the amplification of the heavy chain variable domain and the light chain variable domain in separate reactions the current approach provides for an increased sensitivity and an increased amount of amplified sequences. The use of gene-specific primer in the polymerase chain reactions enhances the specificity and accuracy of the method.

More complex gene structure in the case of human IgG requires a different strategy for the primer design, the placement and the polymerase chain reaction for the required sensitivity and accuracy.

Thus, herein is reported a multiplex real-time reverse transcriptase polymerase chain reaction that can be carried out either without or with the linkage of the heavy and light chain encoding regions that are amplified. For the in vitro translation of the obtained nucleic acids it is beneficial that the encoded domains comprise cysteine residues suitable for the formation of interchain disulfide bonds.

Methods and techniques known to a person skilled in the art, which are useful for carrying out the current invention, are reported e.g. in Ausubel, F. M., ed., Current Protocols in Molecular Biology, Volumes I to III (1997), Wiley and Sons; Sambrook, J., et al., Molecular Cloning: A Laboratory Manual, Second Edition, Cold Spring Harbor Laboratory Press, Cold Spring Harbor, N.Y. (1989); Morrison, S. L., et al., Proc. Natl. Acad. Sci. USA 81 (1984) 6851-6855; U.S. Pat. No. 5,202,238 and U.S. Pat. No. 5,204,244.

The term “immunoglobulin” denotes a protein consisting of one or more polypeptide(s) substantially encoded by immunoglobulin genes. The recognized immunoglobulin genes include the different constant region genes as well as the myriad immunoglobulin variable region genes. Immunoglobulins may exist in a variety of formats, including, for example, Fv, Fab, and F(ab)₂ as well as single chains (scFv) or diabodies. An immunoglobulin in general comprises two so called light chain polypeptides (light chain) and two so called heavy chain polypeptides (heavy chain). Each of the heavy and light chain polypeptides contains a variable domain (variable region) (generally the amino terminal portion of the polypeptide chain) comprising binding regions that are able to interact with a binding partner, generally the antigen. Each of the heavy and light chain polypeptides comprises a constant region (generally the carboxyl terminal portion). The constant region of the heavy chain mediates the binding of the antibody i) to cells bearing a Fc gamma receptor (FcγR), such as phagocytic cells, or ii) to cells bearing the neonatal Fc receptor (FcRn) also known as Brambell receptor. It also mediates the binding to some factors including factors of the classical complement system such as component (C1q). The variable domain of an immunoglobulin's light or heavy chain in turn comprises different segments, i.e. four framework regions (FR) and three hypervariable regions (CDR).

The term “chimeric immunoglobulin” denotes an immunoglobulin, preferably a monoclonal immunoglobulin, comprising a variable domain, i.e. binding region, from a first non-human species and at least a portion of a constant region derived from a second different source or species. Chimeric immunoglobulins are generally prepared by recombinant DNA techniques. In one embodiment chimeric immunoglobulins comprise a mouse, rat, hamster, rabbit, or sheep variable domain and a human constant region. In one embodiment the human heavy chain constant region is a human IgG constant region. In another embodiment the human light chain constant domain is a kappa light chain constant domain or a lambda light chain constant domain.

The “Fc part” of an immunoglobulin is not directly involved in binding to the antigen, but exhibit various effector functions. Depending on the amino acid sequence of the constant region of the heavy chain, immunoglobulins are divided in the classes: IgA, IgD, IgE, IgG, and IgM. Some of these classes are further divided into subclasses, i.e. IgG in IgG1, IgG2, IgG3, and IgG4, or IgA in IgA1 and IgA2. According to the immunoglobulin class to which an immunoglobulin belongs the heavy chain constant regions of immunoglobulins are called α (IgA), δ (IgD), ε (IgE), γ (IgG), and μ (IgM), respectively. The immunoglobulin belongs in one embodiment to the IgG class. An “Fc part of an immunoglobulin” is a term well known to the skilled artisan and defined on basis of the papain cleavage of immunoglobulins. In one embodiment the immunoglobulin contains as Fc part a human Fc part or an Fc part derived from human origin. In a further embodiment the Fc part is either an Fc part of a human immunoglobulin of the subclass IgG4 or IgG1 or is an Fc part of a human immunoglobulin of the subclass IgG1, IgG2, or IgG3, which is modified in such a way that no Fcγ receptor (e.g. FcγRIIIa) binding and/or no C1q binding as defined below can be detected. In one embodiment the Fc part is a human Fc part, in another embodiment a human IgG4 or IgG1 subclass Fc part or a mutated Fc part from human IgG1 subclass. In a further embodiment the Fc part is from human IgG1 subclass with mutations L234A and L235A. While IgG4 shows reduced Fcγ receptor (FcγRIIIa) binding, immunoglobulins of other IgG subclasses show strong binding. However Pro238, Asp265, Asp270, Asn297 (loss of Fc carbohydrate), Pro329, Leu234, Leu235, Gly236, Gly237, Ile253, Ser254, Lys288, Thr307, Gln311, Asn434, or/and His435 are residues which, if altered, provide also reduced Fcγ receptor binding (Shields, R. L., et al., J. Biol. Chem. 276 (2001) 6591-6604; Lund, J., et al., FASEB J. 9 (1995) 115-119; Morgan, A., et al., Immunol. 86 (1995) 319-324; EP 0 307 434). In one embodiment the immunoglobulin is in regard to Fcγ receptor binding of IgG4 or IgG1 subclass or of IgG1 or IgG2 subclass, with a mutation in L234, L235, and/or D265, and/or contains the PVA236 mutation. In another embodiment the mutations are S228P, L234A, L235A, L235E, and/or PVA236 (PVA236 means that the amino acid sequence ELLG (given in one letter amino acid code) from amino acid position 233 to 236 of IgG1 or EFLG of IgG4 is replaced by PVA). In a further embodiment the mutations are S228P of IgG4, and L234A and L235A of IgG1. In one embodiment the heavy chain constant region has an amino acid sequences of SEQ ID NO: 01, or SEQ ID NO: 02, or SEQ ID NO: 01 with mutations L234A and L235A, or SEQ ID NO: 02 with mutation S228P, and the light chain constant region has an amino acid sequence of SEQ ID NO: 03 or SEQ ID NO: 04.

The term “human immunoglobulin” as used herein, denotes an immunoglobulin having variable and constant regions (domains) derived from human germ line immunoglobulin sequences and having high sequence similarity or identity with these germ line sequences. The constant regions of the antibody are constant regions of human IgG1 or IgG4 type or a variant thereof. Such regions can be allotypic and are described by, e.g., Johnson, G. and Wu, T. T., Nucleic Acids Res. 28 (2000) 214-218, and the databases referenced therein.

The term “recombinant immunoglobulin” as used herein denotes an immunoglobulin that is prepared, expressed, or created by recombinant means. The term includes immunoglobulins isolated from host cells, such as E. coli, NS0, BHK, or CHO cells. “Recombinant human immunoglobulins” according to the invention have in one embodiment variable and constant regions in a rearranged form. The recombinant human immunoglobulins have been subjected to in vivo somatic hypermutation. Thus, the amino acid sequences of the VH and VL regions of the recombinant human immunoglobulins are sequences that can be assigned to defined human germ line VH and VL sequences, but may not naturally exist within the human antibody germ line repertoire in vivo.

The term “monoclonal immunoglobulin” denotes an immunoglobulin obtained from a population of substantially homogeneous immunoglobulins, i.e. the individual immunoglobulins of the population are identical except for naturally occurring mutations that may be present in minor amounts. Monoclonal immunoglobulins are highly specific, being directed against a single antigenic site. Furthermore, in contrast to polyclonal immunoglobulin preparations, which include different immuno globulins directed against different antigenic sites (determinants or epitopes), each monoclonal immunoglobulin is directed against a single antigenic site. In addition to their specificity, the monoclonal immunoglobulins are advantageous in that they may be synthesized uncontaminated by other immunoglobulins. The modifier “monoclonal” indicates the character of the immunoglobulin as being obtained from a substantially homogeneous population of immunoglobulins and is not to be construed as requiring production of the immunoglobulin by any particular method.

The term “variable domain” (variable domain of a light chain (V_{L}), variable domain of a heavy chain (V_{H})) as used herein denotes each of the individual domains of a pair of light and heavy chains of an immunoglobulin which are directly involved in the binding of the target antigen. The variable domains are generally the N-terminal domains of light and heavy chains. The variable domains of the light and heavy chain have the same general structure, i.e. they possess an “immunoglobulin framework”, and each domain comprises four “framework regions” (FR), whose sequences are widely conserved, connected by three “hypervariable regions” (or “complementarity determining regions”, CDRs). The terms “complementary determining region” (CDR) or “hypervariable region” (HVR), which are used interchangeably within the current application, denote the amino acid residues of an antibody which are mainly involved in antigen-binding. “Framework” regions (FR) are those variable domain regions other than the hypervariable regions. Therefore, the light and heavy chain variable domains of an immunoglobulin comprise from N- to C-terminus the regions FR1, CDR1, FR2, CDR2, FR3, CDR3, and FR4. CDR and FR amino acid residues are determined according to the standard definition of Kabat, E. A., et al., Sequences of Proteins of Immunological Interest, 5th ed., Public Health Service, National Institutes of Health, Bethesda, Md. (1991).

The term “amino acid” as used within this application denotes the group of carboxy α-amino acids, which directly or in form of a precursor can be encoded by nucleic acid. The individual amino acids are encoded by nucleic acids consisting of three nucleotides, so called codons or base-triplets. Each amino acid is encoded by at least one codon. The encoding of the same amino acid by different codons is known as “degeneration of the genetic code”. The term “amino acid” as used within this application denotes the naturally occurring carboxy α-amino acids and comprises alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

A “nucleic acid” or a “nucleic acid sequence”, which terms are used interchangeably within this application, refers to a polymeric molecule consisting of the individual nucleotides (also called bases) ‘a’, ‘c’, ‘g’, and T (or ‘u’ in RNA), i.e. to DNA, RNA, or modifications thereof. This polynucleotide molecule can be a naturally occurring polynucleotide molecule or a synthetic polynucleotide molecule or a combination of one or more naturally occurring polynucleotide molecules with one or more synthetic polynucleotide molecules. Also encompassed by this definition are naturally occurring polynucleotide molecules in which one or more nucleotides are changed (e.g. by mutagenesis), deleted, or added. A nucleic acid can either be isolated, or integrated in another nucleic acid, e.g. in an expression cassette, a plasmid, or the chromosome of a host cell. A nucleic acid is characterized by its nucleic acid sequence consisting of individual nucleotides.

To a person skilled in the art procedures and methods are well known to convert an amino acid sequence, e.g. of a polypeptide, into a corresponding nucleic acid sequence encoding this amino acid sequence. Therefore, a nucleic acid is characterized by its nucleic acid sequence consisting of individual nucleotides and likewise by the amino acid sequence of a polypeptide encoded thereby.

A nucleic acid encoding a monoclonal immunoglobulin can be obtained from a single cell with a method as reported herein comprising a one tube real-time reverse-transcriptase gene-specific polymerase chain reaction (PCR). Additionally, with a combination of a PCR method as reported herein and an in vitro translation the nucleic acid encoding a monoclonal immunoglobulin can be obtained from a single cell and the encoded immunoglobulin can be provided at least as Fab fragment in quantities sufficient for the characterization of the immunoglobulin's binding properties. In order to amplify the very low amount of mRNA obtained from a single cell, the PCR (polymerase chain reaction) has to be very sensitive.

Thus, based on the amplification of nucleic acid encoding cognate IgG HC (immunoglobulin G heavy chain) and IgG LC (immunoglobulin G light chain) of an IgG isotype immunoglobulin from a single cell with subsequent in vitro translation of the obtained amplified nucleic acid Fab fragments or complete immunoglobulins can be provided. With this method a high sensitive method for obtaining information about an immunoglobulin produced by a single cell is provided. This is possible even from the minute amounts of mRNA of a single cell. The method according to the invention allows for the biochemical characterization of the binding characteristics of an immunoglobulin expressed by a single. Thus, with this method characterization of a higher diversity as opposed to the hybridoma technology can be achieved. Furthermore, as cognate immunoglobulin chains can be obtained e.g. from mature B-cells after antigen contact, selectively the nucleic acids encoding high specific and correctly assembled immunoglobulins can be obtained.

The method as reported herein for obtaining the nucleic acid encoding an immunoglobulin Fab fragment form a single cell comprises a one tube real-time multiplex semi-nested PCR for the amplification of cognate IgG HC and IgG LC encoding nucleic acids (human IgG isotype) from a single B-cell. Thereafter the Fab-fragment can be translated in vitro using an E. coli cell lysate. The expression can be confirmed using ELISA and Western blot methods.

In general the methods as reported herein comprise the following general steps
i) isolating with magnetic micro-beads coated with human CD19 B-cells from peripheral blood,
ii) depositing single cells e.g. by limited dilution or FACS,
iii) extracting the mRNA of the individualized B-cells,
iv) obtaining one or more nucleic acids encoding at least the variable domains (VH and VL) of the immunoglobulin produced by the individualized B-cell,
v) translating in vitro a RNA template, and,
vi) optionally, characterizing the binding properties of the immunoglobulin or immunoglobulin fragment.

The PCR-based approaches as reported herein are highly sensitive and result in high recovery of the amplified nucleic acids encoding the immunoglobulin's heavy and light chains or fragments thereof. Also provided is a method for the expression of functional and stable Fab fragments after in vitro translation of nucleic acid obtained with the PCR-based methods as reported herein.

The terms “polymerase chain reaction” and “PCR”, which can be used interchangeably, denote a method for specifically amplifying a region of nucleic acids, e.g. of DNA or RNA. This method has been developed by K. Mullis (see e.g. Winkler, M. E., et al., Proc. Natl. Acad. Sci. USA 79 (1982) 2181-2185). The region can be a single gene, a part of a gene, a coding or a non-coding sequence. Most PCR methods typically amplify DNA fragments of hundreds of base pairs (bp), although some techniques allow for amplification of fragments up to 40 kilo base pairs (kb) in size. A basic PCR set up requires several components and reagents. These components include a nucleic acid template that contains the region to be amplified, two primer complementary to the 5′- and 3′-end of the region to be amplified, a polymerase, such as Taq polymerase or another thermostable polymerase, deoxynucleotide triphosphates (dNTPs) from which the polymerase synthesizes a new strand, a buffer solution providing a suitable chemical environment for optimum activity and stability of the polymerase, divalent cations, generally Mg²⁺, and finally, monovalent cations like potassium ions.

The terms “multiplex polymerase chain reaction” or “multiplex PCR”, which can be used interchangeably, denote a polymerase chain reaction employing multiple, unique primer in a single PCR reaction/mixture to produce amplicons of varying sizes specific to different DNA sequences. By targeting multiple genes at once, additional information can be obtained from a single test run that otherwise would require several times the reagents and more time to perform. Annealing temperatures for each primer sets must be optimized to work correctly within a single reaction. Besides, amplicon sizes should be different enough to form distinct bands when visualized by gel electrophoresis.

In the human genome the chromosomal loci containing the immunoglobulin encoding genes are located on chromosomes 2, 14, and 22 (see FIG. 1). The human immunoglobulin G heavy chain locus can be found on chromosome 14 (14q32.2) with the chromosomal orientation in the locus: telomere-5′-end-V_{H}-D-J_{H}-C_{H}-3′-end-centromere. The V_{H} segments on the chromosome are classified as depicted in the following Table 1.

TABLE 1
Grouping of the V_{H}-genes into V_{H} families according to Matsuda,
F., et al., J. Exp. Med. 188 (1998) 2151-2162 and Tomlinson,
I. M., et al., V Base sequence directory 1999.

| | Number of family | Genes with open reading |
|---|---|---|
| V_{H} family | members | frame |
| V_{H}1 | 14 | 9/11 |
| V_{H}2 | 4 | 3 |
| V_{H}3 | 65 | 22 |
| V_{H}4 | 32 | 7/11 |
| V_{H}5 | 2 | 2 |
| V_{H}6 | 1 | 1 |
| V_{H}7 | 5 | 1 |

The human immunoglobulin G heavy chain locus comprises overall 123-129 V_{H}-genes, of which 51 are functional, 23 functional D-genes (D=diversity), grouped in seven families, 6 functional J_{H}-genes (J=joining) and in the most frequent haplotype 9 functional C_{H}-genes (C=constant).

The locus for the human immunoglobulin G light chains of the types kappa (κ) and lambda (λ) is located on two different chromosomes, chromosomes 2 and 22. The kappa light chain locus can be found on the short arm of chromosome 2 (2p11.2) and comprises 40 functional V_{κ}-gene segments. These are grouped in seven families. The locus also comprises 5 J_{κ}-genes and a single C_{κ}-gene (Schable, K. F. and Zachau, H. G., Biol. Chem. Hoppe Seyler 374 (1993) 1001-1022; Lefranc, M. P., Exp. Clin. Immunogenet. 18 (2001) 161-174).

TABLE 2
Grouping of the V_{κ}-genes into V_{κ} families according to Foster,
S. J., et al., J. Clin. Invest. 99 (1997) 1614-1627.

| | Number of |
|---|---|
| V_{κ} family | functional genes |
| V_{κ}1 | 19 |
| V_{κ}2 | 9 |
| V_{κ}3 | 7 |
| V_{κ}4 | 1 |
| V_{κ}5 | 1 |
| V_{κ}6 | 3 |

The lambda light chain locus can be found on the long arm of chromosome 22 (22p11.2) and comprises 73-74 V_{λ}-gene of which 30 are functional. These are grouped in ten families which in addition are grouped in three clusters. The locus also comprises 7 J_{λ}-genes, of which 5 are functional.

TABLE 3
Grouping of the V_{λ}-genes into V_{λ} families according
to Frippiat, J. P., et al., Hum. Mol. Genet. 4 (1995) 983-991;
Farner, N. L., et al., J. Immunol. 162 (1999) 2137-2145; Lefranc,
M. P., Exp. Clin. Immunogenet. 18 (2001) 242-254.

| | Number of | |
|---|---|---|
| V_{λ} family | functional genes | Cluster |
| V_{λ}1 | 5 | B |
| V_{λ}2 | 5 | A |
| V_{λ}3 | 8 | A |
| V_{λ}4 | 3 | A-C |
| V_{λ}5 | 3 | B |
| V_{λ}6 | 1 | C |
| V_{λ}7 | 2 | B |
| V_{λ}8 | 1 | C |
| V_{λ}9 | 1 | B |
| V_{λ}10 | 1 | C |

The PCR-based amplification of the nucleic acid encoding an IgG HC and LC or at least the variable domain thereof from a single immunoglobulin producing cell, e.g. from a single B-cell, is based on the single cell deposition of B-lymphocytes followed by a PCR based nucleic acid amplification with specific primer for the variable domain of the heavy and light chain. The outcome of the PCR is essentially depending on the employed PCR primer. At best the employed primer should cover all V-genes, should not be prone to dimer formation and should specifically bind to the cDNA encoding the immunoglobulin. Thus, in one embodiment the nucleic acid encoding an immunoglobulin variable domain is obtained from cDNA.

Due to the large number of functional genes on the human immunoglobulin G locus it is necessary to employ different primer in the PCR reaction in order to cover as many known genes as possible. Therefore, a set of degenerated primer has been established which is also an aspect of the current invention. In one embodiment the amplification of the nucleic acid encoding the heavy and light chain is performed in one polymerase chain reaction. In this embodiment the primer are chosen in order to provide for the amplification of nucleic acids of approximately the same length in order to allow for the same PCR conditions. In this embodiment primer for the nucleic acid encoding the heavy chain are employed whereof one is binding in the heavy chain C_{H}1 region, thus, providing for a nucleic acid fragment of comparable size to that of the corresponding nucleic acid encoding the light chain.

In the methods as reported herein the nucleic acid encoding the light chain variable domain and nucleic acid encoding the heavy chain variable domain are obtained in a single polymerase chain reaction by a combination of the different 5′- and 3′-primer in a single multiplex polymerase chain reaction.

Another aspect of the current invention is a method for obtaining a nucleic acid encoding at least an immunoglobulin variable domain from a single cell comprising the following step:
performing a reverse transcription and polymerase chain reaction in one step with a set of primer comprising two 5′-primer and two 3′-primer and two TaqMan probes.

In one embodiment of this method the 5′-primer employed in the multiplex real-time one tube reverse transcription gene specific primer polymerase chain reaction binds in the coding region for the first framework region of the immunoglobulin. In another embodiment the primer employed in the PCR reaction provide for overhangs encoding the translational start codon ATG for the 5′-primer and/or the translational stop codon TTA for the 3′-primer. This overhang can be useful in an optional following overlapping polymerase chain reaction for the generation of nucleic acids for the in vitro translation of the obtained nucleic acid. In one embodiment this method is for obtaining an immunoglobulin heavy chain variable domain. In one embodiment the immunoglobulin variable domain is an immunoglobulin heavy chain variable domain or an immunoglobulin kappa light chain variable domain or an immunoglobulin lambda light chain variable domain.

In one embodiment the primer employed in the multiplex one tube real-time PCR for obtaining a nucleic acid encoding an immunoglobulin heavy chain variable domain have the nucleic acid sequence of SEQ ID NO: 05 and 06.

TABLE 4
Primer employed in the multiplex real-time PCR
reaction for obtaining a nucleic acid encoding
an immunoglobulin heavy chain variable domain.

| Primer | | | SEQ ID |
|---|---|---|---|
| description | Sequence | Denotation | NO: |
| V_{H} primer | CTTTAAGAAGGA | V_{H}-lfp | 05 |
| binding in the | GATATACCATGG | | |
| FR1 coding | AGGTGCAGCTGK | | |
| region | TGSAGTCTGS | | |
| primer binding | ATCGTATGGGTAG | V_{H}-rfp | 06 |
| in the constant | CTGGTCCCTTAAA | | |
| region coding | CTBTCTTGTCCAC | | |
| region | CTTGGTGTTG | | |

In one embodiment of the methods according to the invention the primer employed in the multiplex one tube real-time PCR for obtaining a nucleic acid encoding an immunoglobulin kappa light chain variable domain have the nucleic acid sequence of SEQ ID NO: 07 and 08.

TABLE 5
Primer employed in the multiplex one tube real-
time PCR for obtaining a nucleic acid encoding
an immunoglobulin kappa light chain variable
domain.

| Primer | | | SEQ ID |
|---|---|---|---|
| description | Sequence | Denotation | NO: |
| V_{κ} primer | CTTTAAGAAGGA | VL(k)-lfp | 07 |
| binding in | GATATACCATGG | | |
| the FR1 | AWRTTGTGMTGA | | |
| coding region | CKCAGTCTCC | | |
| primer binding | ATCGTATGGGTA | VL(k)-rfp | 08 |
| in the constant | GCTGGTCCCTTA | | |
| region coding | ACACTCTCCCCT | | |
| region | GTTGAAGCTC | | |

In one embodiment of the methods according to the invention the TaqMan probes employed in the multiplex one tube real-time PCR for quantitating the PCR result have the nucleic acid sequence of SEQ ID NO: 09 and 10.

TABLE 6
TaqMan probes employed in the multiplex one tube
real-time PCR for obtaining a nucleic acid
encoding immunoglobulin variable domains.

| Primer | | | SEQ ID |
|---|---|---|---|
| description | Sequence | Denotation | NO: |
| TaqMan | Cyan500-CCAAGCTGCTG | IgH | 09 |
| Probe IgH | GAGGGCACGGTCACC-BBQ | | |
| TaqMan | Cy5-CCTTGCTGTCCTGCT | IgL | 10 |
| Probe IgL | CTGTGACACTC--BBQ | | |

With the combination of the PCR method as reported herein and a cell-free in vitro translation system the nucleic acids encoding the cognate immunoglobulin VH and VL domains can be obtained as Fab fragment in quantities sufficient for the characterization of the immunoglobulin's binding properties. In order to amplify the very low amount of mRNA obtained from a single cell, the PCR (polymerase chain reaction) has to be very sensitive.

The term “cell-free in vitro translation system” denotes a cell-free lysate of a prokaryotic or eukaryotic, preferably of a prokaryotic, cell containing ribosomes, tRNA, ATP, CGTP, nucleotides, and amino acids. In one embodiment the prokaryote is E. coli.

Cell-free in vitro translation is a method which has been known in the state of the art for a long time. Spirin et al. developed in 1988 a continuous-flow cell-free (CFCF) translation and coupled transcription/translation system in which a relatively high amount of protein synthesis occurs (Spirin, A. S., et al., Science 242 (1988) 1162-1164). For such cell-free in vitro translation, cell lysates containing ribosomes were used for translation or transcription/translation. Such cell-free extracts from E. coli were developed by, for example, Zubay (Zubay, G., et al., Ann. Rev. Genetics 7 (1973) 267-287) and were used by Pratt (Pratt, J. M., et al., Nucleic Acids Research 9 (1981) 4459-4474; and Pratt, J. M., et al., Transcription and Translation: A Practical Approach, Hames and Higgins (eds.), 179-209, IRL Press (1984)). Further developments of the cell-free protein synthesis are reported in U.S. Pat. No. 5,478,730, U.S. Pat. No. 5,571,690, EP 0 932 664, WO 99/50436, WO 00/58493, and WO 00/55353. Eukaryotic cell-free expression systems are reported by, for example, Skup, D. and Millward, S., Nucleic Acids Research 4 (1977) 3581-3587; Fresno, M., et al., Eur. J. Biochem. 68 (1976) 355-364; Pelham, H. R. and Jackson, R. J., Eur. J. Biochem. 67 (1976) 247-256 and in WO 98/31827.

Based on the amplification of nucleic acid encoding cognate IgG HC (immunoglobulin G heavy chain) and IgG LC (immunoglobulin G light chain) encoding nucleic acids of an IgG isotype immunoglobulin from a single cell and the subsequent in vitro translation of the obtained nucleic acids Fab fragments of the immunoglobulin can be obtained and a high sensitive method for obtaining information about an immunoglobulin produced by a single cell from the minute amounts of mRNA obtainable can be provided. The methods as reported herein permit the characterization of the immunoglobulin of a single B-cell, thus, providing higher diversity as opposed to the hybridoma technology. Furthermore, since the cognate immunoglobulin variable domains or immunoglobulin chains can be obtained from mature B-cells after antigen contact, selectively the nucleic acid encoding high specific and correctly assembled immunoglobulins can be obtained.

Therefore, one aspect of the current invention is a method for producing an immunoglobulin Fab fragment comprising the following steps:
providing a single immunoglobulin producing cell,
obtaining from the cell the nucleic acid encoding the immunoglobulin light and heavy chain variable domains, optionally also encoding a part of the light chain constant domain and a part of the heavy chain C_{H}1 domain, with a one tube real-time multiplex reverse-transcriptase PCR as reported herein,
optionally generating a linear expression matrix comprising the obtained nucleic acids,
translating in vitro the nucleic acids and thereby producing the immunoglobulin Fab fragment.

In one embodiment the translating is by incubating the nucleic acid in vitro with an E. coli cell lysate.

For the recombinant production of an immunoglobulin comprising the variable domains obtained from a single cell with a method according to the invention the obtained nucleic acids encoding the variable domain of the light and heavy immunoglobulin chain can be further modified. For example, the nucleic acid encoding the variable domain can be combined with a nucleic acid encoding an immunoglobulin constant region or a fragment thereof. In one embodiment the nucleic acid encoding the light chain variable domain is combined with a nucleic acid encoding human kappa light chain constant domain of SEQ ID NO: 03 or with a nucleic acid encoding human lambda light chain variable domain of SEQ ID NO: 04. In another embodiment the nucleic acid encoding the heavy chain variable domain is combined with a nucleic acid encoding human immunoglobulin G1 (IgG1) constant region of SEQ ID NO: 01 or with a nucleic acid encoding human immunoglobulin G4 (IgG4) constant region of SEQ ID NO: 02.

The nucleic acid molecules encoding the complete immunoglobulin heavy and light chain or a fragment thereof are in the following referred to as structural genes.

They can be located on the same expression plasmid or can be located on different expression plasmids. The assembly of the complete immunoglobulin or Fab-fragment takes place inside the expressing cell before the secretion of the immunoglobulin to the cultivation medium. Therefore, the nucleic acid molecules encoding the immunoglobulin chains are in one embodiment expressed in the same host cell. If after recombinant expression a mixture of immunoglobulins is obtained, these can be separated and purified by methods known to a person skilled in the art. These methods are well established and widespread used for immunoglobulin purification and are employed either alone or in combination. Such methods are, for example, affinity chromatography using microbial-derived proteins (e.g. protein A or protein G affinity chromatography), ion exchange chromatography (e.g. cation exchange (carboxymethyl resins), anion exchange (amino ethyl resins) and mixed-mode exchange chromatography), thiophilic adsorption (e.g. with beta-mercaptoethanol and other SH ligands), hydrophobic interaction or aromatic adsorption chromatography (e.g. with phenyl-sepharose, aza-arenophilic resins, or m-aminophenylboronic acid), metal chelate affinity chromatography (e.g. with Ni(II)- and Cu(II)-affinity material), size exclusion chromatography, and preparative electrophoretic methods (such as gel electrophoresis, capillary electrophoresis) (Vijayalakshmi, M. A., Appl. Biochem. Biotech. 75 (1998) 93-102).

“Operably linked” refers to a juxtaposition of two or more components, wherein the components so described are in a relationship permitting them to function in their intended manner. The term “linking . . . in operable form” denotes the combination of two or more individual nucleic acids in a way that the individual nucleic acids are operably linked in the final nucleic acid. For example, a promoter and/or enhancer are operably linked to a coding sequence, if it acts in cis to control or modulate the transcription of the linked sequence. Generally, but not necessarily, the DNA sequences that are “operably linked” are contiguous and, where necessary to join two protein encoding regions such as first domain and a second domain, e.g. an immunoglobulin variable domain and an immunoglobulin constant domain or constant region, contiguous and in (reading) frame. A translation stop codon is operably linked to an exonic nucleic acid sequence if it is located at the downstream end (3′-end) of the coding sequence such that translation proceeds through the coding sequence to the stop codon and is terminated there. Linking is accomplished by recombinant methods known in the art, e.g., using PCR methodology and/or by ligation at convenient restriction sites. If convenient restriction sites do not exist, then synthetic oligonucleotide adaptors or linkers are used in accord with conventional practice.

Thus, one aspect of the current invention is a method for producing an immunoglobulin comprising the following steps:
providing a single immunoglobulin producing cell,
obtaining from this cell the nucleic acid encoding the immunoglobulin light and heavy chain variable domains with a method as reported herein,
linking the nucleic acid encoding the light chain variable domain with a nucleic acid encoding an immunoglobulin light chain constant domain of SEQ ID NO: 03 or SEQ ID NO: 04 in operable form and linking the nucleic acid encoding the heavy chain variable domain with a nucleic acid encoding an immunoglobulin heavy chain constant region of SEQ ID NO: 01 or SEQ ID NO: 02 in operable form,
transfecting a eukaryotic or prokaryotic cell with the nucleic acids of the previous step,
cultivating the transfected cell under conditions suitable for the expression of the immunoglobulin,
recovering the immunoglobulin from the cell or the cultivation medium and thereby producing an immunoglobulin.

The term “under conditions suitable for the expression of” denotes conditions which are used for the cultivation of a cell capable of expressing a heterologous polypeptide and which are known to or can easily be determined by a person skilled in the art. It is known to a person skilled in the art that these conditions may vary depending on the type of cell cultivated and type of polypeptide expressed. In general the cell is cultivated at a temperature, e.g. between 20° C. and 40° C., and for a period of time sufficient to allow effective production of the conjugate, e.g. for of from 4 days to 28 days, in a volume of 0.01 liter to 10⁷ liter.

The following examples, sequence listing and figures are provided to aid the understanding of the present invention, the true scope of which is set forth in the appended claims. It is understood that modifications can be made in the procedures set forth without departing from the spirit of the invention.

### DESCRIPTION OF THE SEQUENCE LISTING

SEQ ID NO: 01 human IgG1 heavy chain constant region
SEQ ID NO: 02 human IgG4 heavy chain constant region
SEQ ID NO: 03 human IgG kappa light chain constant domain
SEQ ID NO: 04 human IgG lambda light chain constant domain

### SEQ ID NO: 05 VH-primer

### SEQ ID NO: 06 VCH-primer

### SEQ ID NO: 07 VL-primer

### SEQ ID NO: 08 VCL-primer

SEQ ID NO: 09 TaqMan probe 1
SEQ ID NO: 10 TaqMan probe 2
SEQ ID NO: 11 Primer 1 for overlapping PCR
SEQ ID NO: 12 Primer 2 for overlapping PCR

### DESCRIPTION OF THE FIGURES

FIG. 1 Chromosomal localization of the human immunoglobulin G heavy chain locus (A), the human immunoglobulin kappa light chain locus (B) and of the human immunoglobulin lambda light chain locus (C).

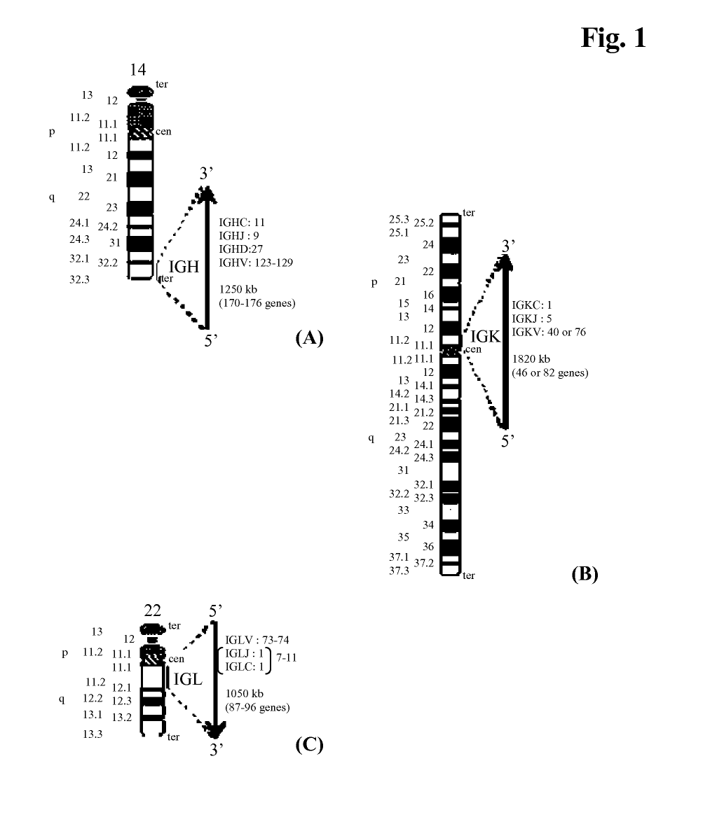

### EXAMPLES

### Materials & Methods

### B-Cells and Plasma Cells:

Samples used in this approach are B-cells and plasma cells isolated from the peripheral blood of healthy donor and tissue (spleen, bone marrow) of transgenic mice for human IgG. Solid tissue is first of all manually disaggregated in DMEM in separate tubes. In the later steps, gentle handling and low temperature minimize cell lysis, which is important for the future positive isolation of the cells of interest and to keep the source of mRNA intact. Disaggregated tissue is suspended by the delicate addition of cell separation media for making of a different cell type gradient (Leucosep-tubes (Greiner Bio-One) with Ficoll density gradient). Suspended cells are purified by centrifugation on the cold separation medium for 20 min. at 800×g and 22° C. in a centrifuge without breaking in order to enrich for plasma cells (PBMC) and lymphocytes. Cells are washed in cold buffer (PBS (phosphate buffered saline), 0.1% (w/v) BSA (bovine serum albumin), 2 mM EDTA (ethylene diamin tetra acetate)) and the supernatant is carefully discarded to keep only the lymphocytes. Lymphocytes are than resuspended in PBS and mixed by carefully pipetting. Centrifugation is effectuated for 5 min. at 800×g and 22° C. to pellet the cells. B-cells and plasma cells are pretreated with murine and human FC blocker to block unspecific binding of Abs on their cells surface. Cells are washed once with buffer (PBS, 0.1% (w/v) BSA, 2 mM EDTA), centrifuged and resuspended in PBS. Only the CD19+ B-cells and CD138+ plasma cells were used. To prevent mRNA degradation an RNAse Inhibitor is added. The positive isolation of the CD19+ B-cells (Dynal Biotech Dynabeads CD19 Pan B) from the mouse spleen has been carried out according to the manufacturer's instructions. The selection of the CD138+ plasma cells (StemCell Technologies EasySep Human CD138 Selection Kit) has been carried out following the manufacturer's instructions.

Separation into Single Cell by the Principle of the Limiting-Dilution Culture or FACS Sorting:

Cells are counted and, by the principle of the limiting-dilution culture, deposited as single cell into the wells of 96-well PCR plates or 384-well plates. Plates are sealed with PCR Film and immediately placed on ice. Sorted cells can be used immediately in RT-PCR (reverse transcriptase polymerase chain reaction) or stored at −20° C. for short-term use or −80° C. for long-term use. Single-cell sorting was performed on a FACSAria cell-sorting system (Becton Dickinson). Cells that stained positive for CD19, highly positive for CD38 and intermediately positive for CD45 were collected and designated plasma cells (PC). Additional gates on forward scatter/side scatter and side scatter width/side scatter height were included to select live lymphocytes and singlets, respectively. Single cells were distributed directly into the wells of 96-well PCR plates (Eppendorf), containing all the necessary PCR reagents in a volume of 10 μl, except for reverse transcriptase, DNA polymerase, buffer and dNTPs and frozen at −80° C. for later processing.

### One Step Multiplex Real-Time Reverse-Transcriptase Gene-Specific PCR:

To be able to amplificate the mRNA in a polymerase chain reaction, B-cells and plasma cells must be distributed directly into the wells of 96-well PCR plates (Eppendorf), containing all the necessary PCR reagents in a volume of 10 μl, except for reverse transcriptase, DNA polymerase, buffer and dNTPs and frozen at −80° C. for later processing.

### RT-Step:

Reverse transcription and PCR were performed in one step (one step Multiplex RT-PCR). The isolated, sorted and stored cells were used as raw material for the reverse transcription or RT-PCR. All necessary reagents were thawed at room temperature. All primer were synthesized in the MOLBIOL TIB GmbH laboratories. The plates and all other reagents were kept on ice during the entire procedure. For cDNA syntheses the gene specific primer with extensions were used directly. The enzyme complex consists of two Sensiscript reverse transcriptases and one Omniscript polymerase (Qiagen OneStep RT PCR). The rewriting of the mRNA into cDNA was performed by the Sensiscript complex (Qiagen OneStep RT PCR) and the amplification of the cDNA was performed using the HotStarTaq DNA Polymerase (Qiagen OneStep RT PCR), which is a chemically form of a recombinant 94 kDa DNA polymerase (deoxynucleoside-triphosphate: DNA deoxynucleotidyltransferase, EC 2.7.7.7), originally isolated from Thermos aquaticus expressed in E. coli. The cells were sorted in a 96-well PCR plate and stored in a volume of 10 μl, containing 5 μl PCR H₂O grade, 1 μl 0.1 μM primer for VH and VL, 1 μl RNAse inhibitor 20 U/reaction and 3 μl Tris 1.5 mM. Before adding the other 10 μl for performing the PCR reaction, the cells stored at −60° C. were briefly centrifuged (20 sec. at 1400 rpm) to collect the liquid and cells on the bottom of the wells.

TABLE 7
Master Mix 1 used for the RT-PCR.

| | Final | volume/well |
|---|---|---|
| Master Mix 1 | concentration/well | (μl) |
| H₂O | | 5 |
| primer V_{H}/VL(k) | 0.1 μM | 1 |
| RNAse Inhibitor | 20 U/reaction | 1 |
| Tris-buffer | 1.5 mM | 3 |
| B/Plasma cells | | |
| final volume | | 10 |

TABLE 8
Master Mix 2 used for the RT-PCR.

| | Final | volume/well |
|---|---|---|
| Master Mix 2 | concentration/well | (μl) |
| H₂O | 1x | 2.2 |
| 5x Buffer | 1x | 4 |
| dNTP 10 mM each | 400 μM each | 0.8 |
| 5x Q-Solution | 0.25x | 1 |
| One Step RT PCR Enzyme | | 1.2 |
| mix | | |
| RNAse Inhibitor | 20 U | 1 |
| final volume | | 10 |

10 μl per well of Master Mix 2 were added to the cells. The second Master Mix contained 2.2 μl H₂O PCR grade, 4 μl of 1× buffer, 0.8 μl of dNTPs 400 μM each, 1 μl of Q-solution 0.25×, 1.2 μl of the enzyme complex and 1 μl of RNAse inhibitor 20U.

TABLE 9
Primer used for the RT-PCR.

| | | |
|---|---|---|
| Ig heavy chain | Ig light chain (κ) | TaqMan |
| primer | primer | probe |

| | | | | | |
|---|---|---|---|---|---|
| V_{H}-lfp | VL(k)-lfp | VL(k)- | SEQ | IgH | SEQ |
| | | lfp | ID NO: | | ID NO: |
| | | | 07 | | 09 |
| V_{H}-rfp | VL(k)-rfp | VL(k)- | SEQ | IgL | SEQ |
| | | rfp | ID NO: | | ID NO: |
| | | | 08 | | 10 |

TABLE 10
Block cycler program for the RT-GSP- PCR.

| Temperature | Time | Step | Cycles |
|---|---|---|---|
| 50° C. | 30 min. | reverse transcription | 1 |
| 95° C. | 15 min. | denaturation | 1 |
| 94° C. | 40 sec. | denaturation | 11 |
| 52° C. | 1 min. | annealing | |
| 72° C. | 1 min. | elongation | |
| 94° C. | 41 sec. | denaturation | 29 |
| 60° C. | 1 min. | annealing | |
| 72° C. | 1 min. | elongation | |
| 72° C. | 10 min. | final elongation | 1 |
| 4° C. | ∞ | cooling | |

### Purification of PCR Products:

To improve the efficiency of the generation of linear template for the in vitro translation in the next overlapping PCR (third PCR) the purification of the previously amplified PCR products was performed by removing unincorporated primer, dNTPs, DNA polymerases and salts used during PCR amplification in order to avoid interference in downstream applications. Agencourt AMPure was used. The buffer is optimized to selectively bind PCR amplicons 100 bp and larger to paramagnetic beads. Excess oligonucleotides, nucleotides, salts, and enzymes can be removed using a simple washing procedure. The resulting purified PCR product is essentially free of contaminants and can be used in the following applications: Fluorescent DNA sequencing (including capillary electrophoresis), microarray spotting, cloning and primer extension genotyping. The work flow for 96-well format started with gently shaking the beads stored in buffer to resuspend any magnetic particle that may have settled. The correct volume of 36 μl of beads solution was added to the 20 μl of sample and the mix was pipetted 10 times up and down. The following step was incubating for 10 minutes and afterwards the reaction plate was placed onto a magnetic plate for 10 minutes to separate beads from solution. The cleared solution (supernatant) was aspirated from the reaction plate and discard. For the beads-cDNA washing 200 μl of 70% ethanol were dispersed per well and incubated at room temperature for at least 30 seconds. The ethanol was aspirated out and discarded. The washing step was performed two times and then the reaction plate was left to air-dry for 20 minutes at room temperature. It followed with the addition of 40 μl of elution buffer and the mix was again pipetted 10 times up and down. After the cDNA dissociation from the magnetic beads, the purified DNA was transferred into a new plate.

### Overlapping Extension PCR:

The amplified DNA was afterwards linked by an overlapping extension PCR method with the following components, necessary for the transcription/translation step: a ribosome binding site (RBS), a T7 promoter and a T7 terminator sequences. For this PCR, 2 μl of the second PCR were taken to a final volume of 20 μl containing: 10.7 μl water, 2 μl of 10× reaction buffer with MgCl₂ (10 mM), 0.8 μl of DMSO, 0.5 μl dNTPs (10 mM each), 1.6 μl T7 promoter and terminator primer (6 μM each), 0.4 μl C-terminal HA-Tag primer and 0.4 μl of enzyme blend, all from the RTS E. coli Linear Template Generation Set, HA-Tag (Roche Diagnostics GmbH, Mannheim, Germany). Finally, the overlapping PCR products were used as template for in vitro transcription using Escherichia coli lysate and the resulting functional Fab was screened against the F(ab′)₂ IgG by enzyme-linked immunoabsorbent assay (ELISA).

TABLE 11
Components used for the PCR.

| | | Final |
|---|---|---|
| Component | Volume (μl) | concentration |
| Water, PCR grade | 10.7 | |
| 10x Reaction Buffer with MgCl₂ (10 mM) | 2 | 1x |
| DMSO | 0.8 | |
| PCR Nucleotide mix (10 mM each) | 0.5 | 250 μM |
| Working solution T7 Prom Primer (6 μM) | 1.6 | 0.48 μM |
| Working solution T7 Term Primer (6 μM) | 1.6 | 0.48 μM |
| Working solution C-term HA-tag (6 μM) | 0.4 | 0.48 μM |
| Enzyme Blend | 0.4 | |
| PCR 2 product | 2 | |
| Final volume | 20 | |

TABLE 12
Block cycler program for the third PCR.

| Temperature (° C.) | Time | Number of cycles |
|---|---|---|
| 95 | 4 min. | 1 |
| 95 | 1 min. | 45 |
| 60 | 1 min. | |
| 72 | 1 min. 30 sec. | |
| 72 | 7 min. | 1 |
| 4 | ∞ | |

### Gel Electrophoresis:

The gel electrophoresis analysis (1% agarose gel, Invitrogen Corp., USA) was performed to evaluate the amplification and the specificity of the cDNA templates with the appropriate controls.

TABLE 13
Gel analysis protocol.

| Component | Volume (μl) | Migration time |
|---|---|---|
| H₂O | 6 | |
| 5x Orange G | 3 | |
| PCR product | 6 | |
| Final volume | 15 | |
| Volume for gel | 10 | 20 min. |

### In Vitro Transcription and Translation:

The in vitro coupled transcription and translation was carried out following the manufacturer's protocol RTS 100 E. coli Disulfide Kit (Roche Diagnostics GmbH, Mannheim, Germany) with components as reported (see Table 12). 4 μl of each overlapping PCR product was transcribed and translated in a total volume of 50 μl, at 37° C. for 20 hours in the RTS Proteo Master Instrument (Roche Diagnostics GmbH, Mannheim, Germany). A control reaction was performed under identical conditions without cDNA template. GFP (green fluorescent protein) vectors were added to the reaction system for autoradiography as positive control. After the in vitro transcription/translation, the 50 μl reaction mixture was transferred in 75 μl PBS (1:2.5 dilution) and incubated at 4° C. overnight for the correct folding and maturation of the protein.

TABLE 14
Components for the in vitro transcription and translation.

| Mix | Component | Volume (μl) |
|---|---|---|
| Mix 1: | E. coli lysate | 25 |
| | Lysate activator | 1 |
| | Final volume | 26 |
| | incubate for 10-20 min. at RT | |
| Mix 2: | Feeding mix | 640 |
| | Amino acid mix | 140 |
| | Methionine | 20 |
| | H₂O | 200 |
| | Final volume | 1000 |
| Mix 3: | Reaction mix | 7 |
| | Amino acid mix | 7 |
| | Methionine | 1 |
| | Mix 1 | 25 |
| | GroE Supplement | 5 |
| | RNAse inhibitor | 1 |
| | PCR 3 product | 4 |
| | Final volume | 50 |

### ELISA:

A 384-well plate (Nunc GmbH & Co. KG, Thermo Fisher Scientific, Langenselbold, Germany) was coated with 50 μl (1:1000 in PBS) goat anti-human IgG Fab fragment (produced by Bethyl Laboratories Inc., obtained from Biomol GmbH, Hamburg, Germany, 1 mg/1 ml) incubated at 4° C. overnight. The plate was washed three times with washing solution (100 μl PBST (phosphate buffered saline Tween-20)) and 60 μl of Blocking solution (0.25% CroteinC (w/v)/0.5% Tween (w/v)/PBS) was added, incubated for 1 h at room temperature. Another washing step (3×100 μl PBST) was performed and 37.5 μl sample was transferred, as well as 37.5 ml negative control (negative control from the in vitro transcription/translation) and 37.5 μl positive control, containing 0.75 μl of human recombinant Fab fragment (Roche Diagnostics GmbH, Mannheim, Germany). The samples were titrated to a 1:3 dilution. The plate was incubated for 1.5 h at room temperature. After a washing step (3×100 μl PBST), 25 μl goat anti-human IgG F(ab′)₂ (Dianova, Hamburg, Germany; 0.8 mg/ml (1:2000 diluted in Blocking Solution)) was added and incubated for 1 h at room temperature. The last washing step (3×100 μl PBST) was performed and 25 μl of TMB (POD Substrate, Roche Diagnostics GmbH, Mannheim, Germany, Art-No: 1 484 281) was pipetted into each well. After 2-3 minutes the absorption signal was detected at 405 nm and 495 nm (Tecan, Safire 2; Tecan Deutschland GmbH, Crailsheim, Germany).

### Flow Cytometric Analysis and Cell Sorting:

For FACS analysis and cell sorting monoclonal antibodies, either biotinylated or conjugated with either FITC (fluorescein isothiocyanate), PE (Phycoerythrin), or APC (allophycocyanine) against the following antigens were used: CD3 (UCHT1), CD4 (13B8.2), CD8 (B9.11), CD40 (MAB89), CD80 (MAB104), CD83 (HB15a), CD86 (HA5.2B7) (all available from Imunotech/Beckman Coulter, Marseille, France), CD19 (HIB19), CD20 (2H7), CD34(581), IL-3Ra/CD123 (9F5), CD11c (B-ly6) CD14 (M5E2), CD24, CD22a, CD38, CD138 (all available from BD Pharmingen, San Diego, Calif., USA), CD45 (HI30), CD45RA (MEM56), HLA-DR (TU36) (all available from Caltag, Burlingame, Calif., USA), TLR2 (TL2.1), TLRR4 (HTAl25), TCRab (IP26), (all available from Bioscience, San Diego, Calif.), BDCA-1, BDCA-2, BDCA-4, CD25 (4E3) (all available from Miltenyi Biotec, Bergisch Gladbach, Germany), IgM (Jackson Immunoresearch, West Grove, Pa., USA), CCR7 (3D12, provided by M. Lipp, Berlin, Germany). The IOTest Beta Mark was used for Vb analysis (Imunotech/Beckman Coulter). Streptavidin conjugated FITC, PE, or APC (all BD Pharmingen) were used for visualization of biotinylated antibodies. Dead cells were excluded by propidium iodide staining Appropriate isotype-matched, irrelevant control mAbs were used to determine the level of background staining Cells were analyzed using a FACS Calibur and sorted using a FACSAria (Becton Dickinson Immunocytometry Systems, Mountain View, Calif., USA).

### Example 1

### Amplification of IgG Genes from Humanized Immunized Mice's Single B Cell by a Real-Time One Tube Reverse-Transcriptase Polymerase Chain Reaction

### Example 2

### Generation of Linear Template for In Vitro Translation

For the first polymerase chain reaction gene specific primer have been designed comprising the necessary overlapping sequences to the regulatory DNA regions of the T7 phage. For the second polymerase chain reaction the product of the first PCR was combined with nucleic acid fragments comprising the regulatory sequences and encoding the tag-sequence, respectively. A 3′-terminal extension was achieved by hybridization with the nucleic acid fragments comprising the regulatory elements. This linear expression construct is further amplified with the help of two terminal primer. These primer comprise the following sequence: 5′-CTTTAAGAAGGAGATATACC+ATG+15-20 bp of the gene-specific sequence (5′-primer, SEQ ID NO: 11) or 5′-ATCGTATGGGTAGCTGGTCCC+TTA+15-20 bp of the gene-specific sequence (3′-primer, SEQ ID NO: 12).

In Figure X lanes 1, 5 and 9 represent the blank water controls. The heavy chain nucleic acid are contained in lanes 4, 8, and 12, and the kappa light chains in lanes 3, 7, and 11. Lanes 2, 6, and 10 show combined samples of both chains. All nucleic acids have the expected size (see Table 38).

TABLE 15
Size of the linear expression constructs.

| immunoglobulin | two fixed primer | one fixed primer | two variable |
|---|---|---|---|
| chain | sets | set | primer sets |
| IgG HC | ~1110 bp | ~1110 bp | ~822 bp |
| IgG LC(κ) | ~1089 bp | ~1089 bp | ~799 bp |

### Example 3

In vitro translation and huFab specific ELISA

In vitro translation is carried out as outlined above.

As can be seem from FIG. 10 nucleic acids obtained with a two-step polymerase chain reaction with two variable primer sets does not provide for a linear expression construct which allows the in vitro production of the encoded Fab immunoglobulin fragment. In contrast the two-step polymerase chain reaction with one fixed and one variable set of primer employed in separated successive polymerase chain reactions allows for the subsequent provision of a linear expression construct and the in vitro translation of IgG HC and IgG LC comprising immunoglobulin Fab fragment.

In contrast to this is the two-step polymerase chain reaction comprising one fixed set of primer more efficient in the multiplex format as the polymerase chain reaction employing two fixed sets of primer. By employing only one fixed set of primer up to 5-times higher optical densities can be achieved.

1-12. (canceled)

13. A method for the amplification and quantification of a cognate pair of IgG heavy and light chains encoding nucleic acids from a single cell comprising the following step:
performing a reverse transcription and polymerase chain reaction in one step with a first and a second 5′-primer and a first and a second 3′-primer and a first and a second TaqMan probe.

14. The method according to claim 13, wherein
a) the first 5′-primer is complementary to a nucleic acid sequence encoding a heavy chain leader peptide or a first heavy chain framework region, and/or
b) the second 5′-primer is complementary to a nucleic acid sequence encoding a light chain leader peptide or a first light chain framework region, and/or
c) the first 3′-primer is complementary to a nucleic acid sequence encoding C-terminal amino acid residues of a heavy chain CH1 domain, and/or
d) the second 3′-primer is complementary to a nucleic acid sequence encoding C-terminal amino acid residues of a light chain constant domain, and/or
e) the first TaqMan probe is complementary to a nucleic acid encoding N terminal amino acid residues of a heavy chain CH1 domain, and/or
f) the second TaqMan probe is complementary to a nucleic acid encoding N-terminal amino acid residues of a light chain constant domain.

15. A method for obtaining a monoclonal antibody comprising the following step
obtaining a nucleic acid encoding an immunoglobulin fragment wherein the nucleic acid is obtained by specific amplification of cDNA fragments obtained from a mRNA of a single immunoglobulin producing cell with the method according to claim 13.

16. The method according to claim 15, further comprising
transcribing the in vitro translated nucleic acid encoding the immunoglobulin fragment to obtain an mRNA, and
translating the mRNA in vitro by employing an E. coli cell lysate.

17. The method according to claim 13, wherein the primers provide for overhangs encoding a translational start codon ATG for the 5′-primers and/or a translational stop codon TTA for the 3′-primers.

18. The method according to claim 15, wherein the primers provide for overhangs encoding a translational start codon ATG for the 5′-primers and/or a translational stop codon TTA for the 3′-primers.

19. The method according to claim 13, further comprising the additional step of:
obtaining an mRNA from the single cell.

20. A method for producing an immunoglobulin Fab-fragment comprising the following steps:
providing a single immunoglobulin producing cell,
obtaining from the cell the nucleic acid encoding an immunoglobulin light and heavy chain variable domains, or encoding a part of a light chain constant domain and a part of a heavy chain CH1 domain with a multiplex one tube real-time reverse-transcriptase gene-specific polymerase chain reaction for the amplification and quantification of the cognate IgG heavy and light chains encoding nucleic acids of claim 1,
generating a linear expression matrix comprising the obtained nucleic acid, and
translating in vitro the nucleic acid and thereby producing the immunoglobulin Fab fragment.

21. A method for producing an immunoglobulin comprising the following steps:
providing a single immunoglobulin producing cell,
obtaining from the cell the nucleic acid encoding the immunoglobulin light and heavy chain variable domains with a multiplex one tube real-time reverse-transcriptase gene-specific polymerase chain reaction for the amplification and quantification of the cognate IgG heavy and light chains encoding nucleic acids of claim 13,
operably linking each of the nucleic acids obtained in the previous step with a nucleic acid encoding the not encoded C-terminal constant domain amino acid residues of the respective immunoglobulin light or heavy chain constant domain,
transfecting a eukaryotic or a prokaryotic cell with the nucleic acids obtained in the previous step,
cultivating the transfected cell, under conditions suitable for the expression of the immunoglobulin, and
recovering the immunoglobulin from the cell or the cultivation medium and thereby producing the immunoglobulin.

22. The method according to claim 15, wherein the immunoglobulin is an immunoglobulin of class G (IgG).

23. The method according to claim 21, wherein the immunoglobulin is an immunoglobulin of class G (IgG).

24. The method according to claim 15, wherein the single cell is a single B-cell or a single plasmablast or a single plasma cell.

25. The method according to claim 21, wherein the single cell is a single B-cell or a single plasmablast or a single plasma cell.

26. A nucleic acid selected from the group consisting of SEQ ID NOs: 05, or 06, or 07, or 08, or 09, or 10.

27. A kit comprising the nucleic acids of claim 26.


SEQUENCE LISTING (12 sequences)

SEQ ID NO: 1 - protein, 330 aa, Homo sapiens [sequence omitted]

SEQ ID NO: 2 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 3 - protein, 107 aa, Homo sapiens [sequence omitted]

SEQ ID NO: 4 - protein, 104 aa, Homo sapiens
PKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 5 - DNA, 46 bp, Artificial Sequence
  VH primer binding in the FR1 coding region
ctttaagaaggagatataccatggaggtgcagctgktgsagtctgs

SEQ ID NO: 6 - DNA, 49 bp, Artificial Sequence
  primer binding in the constant region coding region
atcgtatgggtagctggtcccttaaactbtcttgtccaccttggtgttg

SEQ ID NO: 7 - DNA, 46 bp, Artificial Sequence
  Vkappa  primer binding in the FR1 coding region
ctttaagaaggagatataccatggawrttgtgmtgackcagtctcc

SEQ ID NO: 8 - DNA, 46 bp, Artificial Sequence
  primer binding in the constant region coding region
atcgtatgggtagctggtcccttaacactctcccctgttgaagctc

SEQ ID NO: 9 - DNA, 26 bp, Artificial Sequence
  TaqMan Probe IgH, nucleotide at position 1 linked to Cyan500 dye, nucleotide at position 26 linked to BBQ dye
ccaagctgctggagggcacggtcacc

SEQ ID NO: 10 - DNA, 26 bp, Artificial Sequence
  TaqMan Probe IgL, nucleotide at position 1 linked to Cy5 dye, nucleotide at position 26 linked to BBQ dye
ccttgctgtcctgctctgtgacactc

SEQ ID NO: 11 - DNA, 23 bp, Artificial Sequence
  Primer 1 for overlapping PCR
ctttaagaaggagatataccatg

SEQ ID NO: 12 - DNA, 24 bp, Artificial Sequence
  Primer 2 for overlapping PCR
atcgtatgggtagctggtccctta